(19) United States
(12) Patent Application Publication
(10) Pub. No.: US 2004/0258645 A1
(43) Pub. Date: Dec. 23, 2004

(54) Means for improving the appearance of mammalian keratinous tissue

(76) Inventors: Amy Violet Trejo, Mason, OH (US); Mark Leslie Kacher, Mason, OH (US); George C. Daher, East Windsor, NJ (US); Catherine Lowman Oppenheimer, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/769,280

(22) Filed: Jan. 30, 2004

Related U.S. Application Data
(60) Provisional application No. 60/444,140, filed on Jan. 31, 2003.

(51) Int. Cl. A61K 7/11; A61K 7/06; A61K 31/7008

(52) U.S. Cl. 424/70.13; 514/62; 514/560; 424/766

(57) ABSTRACT

A personal care kit comprising separate containers packaged together in a unitary form comprising a product suitable for oral consumption and a topical personal care product and a method of improving the condition of skin and/or hair by following a regimen utilizing the kit of the present invention.

### CROSS REFERENCE TO RELATED APPLICATION(S)

This application claims the benefit of U.S. Provisional Application No. 60/444,140, filed Jan. 30, 2003.

### TECHNICAL FIELD

The present invention relates to a personal care kit containing a combination of dietary supplements, particularly essential fatty acids, sugar amines, antioxidants, pro-biotics, vitamins, and/or optionally micronutrient metals in combination with a product suitable for topical application to the skin and/or hair.

### BACKGROUND

Many personal care products currently available to consumers are directed primarily to improving the health and/or physical appearance of the skin and/or hair. Among these products, many focus directly on improving the external conditions and outer appearance of the skin and/or hair. Exemplary product forms include solid bars, lotions, shampoos, gels, creams, etc. No matter what the form, however, most forms attempt to achieve the same goal of providing the user with a mild, non-irritating, non-drying, invigorating, clean-feeling bathing or conditioning experience. Ideally, such benefits are derived in conjunction with additional aesthetic benefits, like moisturization or some other type of therapeutic treatment.

Skin and hair are subject to insults by many extrinsic and intrinsic factors. Extrinsic factors include environmental pollution, wind, heat, low humidity, ultraviolet radiation (e.g., from sun exposure), and the like. Intrinsic factors include chronological aging and other biochemical changes produced by the body. Additionally, diet and nutrition have a major impact on these extrinsic and intrinsic factors that can affect the natural appearance of skin and hair. In today's society, people commonly consume foods rich in fat and cholesterol. Consequently, a number of people are deficient and imbalanced in a variety of nutrients. This combination of extrinsic and intrinsic factors with improper diet can account for an overall lack of feeling and looking one's best.

Numerous products have been described in the art as being useful for regulating and/or improving skin and/or hair conditions via external applications of a composition. For example, cosmetic creams and moisturizers are used to provide skin care benefits for the treatment of conditions such as wrinkles, fine lines, acne, and other forms of undesirable skin surface textures. Shampoos and conditioners are used to improve the texture, feel, hair strand alignment (e.g., frizziness), ease of combing, and other hair care benefits. Although these products may provide external aesthetic benefits, improper nutrition can prevent consumers from obtaining the optimal results they so desire.

Based on the foregoing, there is a continuing need to provide personal care products that not only provide external improvements to skin and/or hair, but also provide the internal, biological nutrients essential for maximizing and optimizing aesthetically-pleasing qualities in the skin and hair. There is also a continuing need to provide such a personal care product that provides characteristics that have become increasingly important to consumers, including efficacy, cost effectiveness, convenience and compactness.

### SUMMARY OF THE INVENTION

It has now been found that dietary supplementation with appropriate anti-oxidants and nutrients can enhance the performance of personal care products to provide consumer-noticeable beauty benefits. By packaging suitable essential nutrients and anti-oxidants in combination with a high quality hair and/or skin care product, consumers can obtain a convenient, economical, and effective personal care product that provides benefits both inside and out. For example, consumption of essential fatty acids, sugar amines (also known as amino sugars), and/or anti-oxidants in combination with the topical application of a skin and/or hair care product, provide enhanced benefits in regulating skin and/or hair conditions previously unrecognized in the art of which the present inventors are aware. Also, for example, consumption of omega-3 fatty acids, glucosamine HCl, and/or tocopherol acetate in combination with the topical application of a skin care and/or hair care product may also synergistically regulate (prophylactically and/or therapeutically) undesirable skin and/or hair textures, including fine lines, wrinkles, enlarged pores, roughness, dryness, hair loss and/or other hair growth abnormalities (i.e., thinning of hair, increased brittleness, etc.), hair strand alignments (e.g., frizziness), dullness, and other forms of damaged skin and/or hair associated with extrinsic and intrinsic factors. Further, for example, consumption of omega-3 fatty acids, iron, vitamin D, zinc, selenium, and copper may also synergistically regulate (prophylactically and/or therapeutically) undesirable skin and/or hair textures, including fine lines, wrinkles, enlarged pores, roughness, dryness, hair loss and/or other hair growth abnormalities (i.e., thinning of hair, increased brittleness, etc.), hair strand alignments (e.g., frizziness), dullness, and other forms of damaged skin and/or hair associated with extrinsic and intrinsic factors.

None of the existing art discloses a package product that provides all of the advantages and benefits of the present invention.

The present invention relates to a personal care kit comprising:

a) a product suitable for oral consumption comprising one or more of:

i) a safe and effective amount of one or more essential fatty acids;

ii) a safe and effective amount of a sugar amine or a pharmaceutically acceptable salt thereof; and

iii) a safe and effective amount of an anti-oxidant.

b) a product suitable for topical application to the skin and/or hair wherein a) and b) are individually contained then packaged together in a unitary form.

The present invention also relates to a method of improving the condition of skin and/or hair by utilizing the personal care kit of the present invention wherein said method comprises the steps of:

a) orally administering about three times a day to a mammal, a product suitable for oral consumption comprising one or more of: a safe and effective amount of one or more essential fatty acids; a safe and effective amount of a sugar amine or a pharmaceutically acceptable salt thereof; and a safe and effective amount of an anti-oxidant;

b) topically applying about two times a day to said mammal a product suitable for application to the skin and/or hair.

These and other features, aspects, and advantages of the present invention will become evident to those skilled in the art from a reading of the present disclosure.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The kit of the present invention can comprise, consist essentially of, or consist of, the components of the present invention as well as other ingredients described herein. As used herein, “consisting essentially of” means that the kit, composition, article, product or component may include additional ingredients, but only if the additional ingredients do not materially alter the basic and novel characteristics of the claimed kit or methods.

All publications cited herein are hereby incorporated by reference in their entirety.

The term “topical application”, as used herein, means to apply or spread a composition included in the present invention onto the surface of mammalian keratinous tissue.

The term “keratinous tissue,” as used herein, refers to keratin-containing layers disposed as the outermost protective covering of mammals (e.g., humans, dogs, cats, etc.) which includes, but is not limited to, skin, mucosa, lips, hair, toenails, fingernails, cuticles, hooves, etc.

The term “safe and effective amount” as used herein means an amount of a compound or composition sufficient to significantly induce a positive benefit, preferably a positive keratinous tissue appearance or feel benefit, including independently or in combinations the benefits disclosed herein, but low enough to avoid serious side effects, i.e., to provide a reasonable benefit to risk ratio, within the scope of sound judgment of the skilled artisan.

The kit of the present invention is useful for regulating keratinous tissue condition. Regulation of keratinous tissue condition is often required due to conditions that may be induced or caused by factors internal and/or external to the body. For instance, “regulating skin condition” includes prophylactically regulating and/or therapeutically regulating skin condition, and may involve one or more of the following benefits: thickening (i.e., building the epidermis and/or dermis layers of the skin and/or the subcutaneous layers such as fat and muscle and where applicable the keratinous layers of the nail and hair shaft) to reduce atrophy (e.g., of the skin), increasing the convolution of the dermal-epidermal border, non-melanin skin discoloration such as under eye circles, blotching (e.g., uneven red coloration due to, e.g., rosacea) (hereinafter referred to as “red blotchiness”), sallowness (pale color), discoloration caused by telangiectasia or spider vessels, discolorations due to melanin (e.g., age spots, uneven pigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like, in the skin which may be detected visually or by feel). As used herein, therapeutically regulating skin condition includes ameliorating, e.g., diminishing, minimizing and/or effacing, discontinuities in skin. Regulating skin condition involves improving skin appearance and/or feel.

As used herein, prophylactically regulating skin condition includes delaying, minimizing and/or preventing visible and/or tactile discontinuities in skin (e.g., texture irregularities in the skin which may be detected visually or by feel).

As used herein, therapeutically regulating skin condition includes ameliorating, e.g., diminishing, minimizing and/or effacing, discontinuities in skin.

As used herein, “regulating hair condition” is intended to include improving the texture, cleanliness, condition, feel, hair strand alignment (e.g., frizziness), ease of combing/brushing, volume, style control, color penetration, and other hair care benefits.

The kit of the present invention is also useful for improving skin and/or hair appearance and/or feel. For example, the kit of the present invention is useful for regulating the appearance of skin condition by providing an overall visual improvement in skin appearance following oral administration of a dietary supplement regimen in combination with the topical application of a suitable product to the skin. Also, for example, the kit of the present invention is useful for regulating the appearance of hair conditions by providing an overall visual improvement in hair appearance following oral administration of a dietary supplement regimen in combination with the topical application of a suitable product to the hair.

The compositions included in the kit of the present invention provide additional benefits, including stability, absence of significant (consumer-unacceptable) skin irritation and good aesthetics, including consumer acceptable appearance (i.e., no unpleasant odor or discoloration present).

The compositions included in the kit of the present invention are stable. The ingredients used herein are compatible with each other and with other skin care actives including, but not limited to, terpene alcohols, retinoids, peptides, phytosterols, tocopherol acetate, and vitamin B₃ compounds. Therefore, the compositions included in the kit are capable of providing additive and/or synergistic skin and/or hair benefits. Additionally, the products included in the kit of the present invention have good product stability and a reasonably long shelf-life.

The resulting compositions included in the kit of the present invention have good aesthetics. Examples of good aesthetics include compositions, such as luxurious creams and lotions, that (i) are light and nongreasy, (ii) have a smooth, silky feel upon the skin and/or hair, (iii) spread easily, (iv) absorb quickly, and/or (v) have no kinesthetic issues (i.e., burn, sting, itch, tingle and the like). Other examples of good aesthetics include compositions that have a consumer acceptable appearance (i.e. no unpleasant odor or discoloration present), and provide good skin and/or hair feel.

The kit of the present invention, including the essential and optional components thereof, is described in detail hereinafter.

Materials

I. Essential Fatty Acids

The kit of the present invention may include a product suitable for oral consumption comprising a safe and effective amount of one or more essential fatty acids. As used herein, “essential fatty acids” refers to fats that are essential to the diet because the body cannot produce them and are classified as either omega-3 fatty acids or omega-6 fatty acids. Preferably, the daily dose is from at least about 0.1 g to about 3 g, more preferably from about 0.75 g to about 2.5 g and, even more preferably, from about 1 g to about 2 g, of the essential fatty acid.

Examples of omega-3 fatty acids that are useful herein include alpha-linolenic acid, stearidonic acid, eicosapentanoic acid, (EPA), docosahexanoic acid (DHA), and mixtures thereof. Preferably, the omega-3 fatty acid is EPA, DHA, and mixtures thereof.

Examples of omega-6 fatty acids that are useful herein include linoleic acid, gamma-linolenic acid, arachidonic acid, and mixtures thereof. Preferably, the omega-6 fatty acid is gamma-linolenic acid.

Additionally, combinations of two or more essential fatty acids may be used.

II. Sugar Amines (Amino Sugars)

The kit of the present invention includes a product suitable for oral consumption comprising a safe and effective amount of a sugar amine or a pharmaceutically acceptable salt thereof. As used herein, “sugar amine” refers to an amine derivative of a six-carbon sugar. Preferably, the daily dose is from about 0.1 g to about 3 g, more preferably from about 0.75 g to about 2.5 g and, even more preferably, from about 1 g to about 2 g, of the sugar amine.

Examples of sugar amines that are useful herein include glucosamine, N-acetyl glucosamine, mannosamine, N-acetyl mannosamine, galactosamine, N-acetyl galactosamine; their derivatives; their salts; and mixtures thereof. Preferably, the sugar amine is glucosamine. Additionally, combinations of two or more sugar amines may be used.

III. Anti-Oxidants

The kit of the present invention includes a product suitable for oral consumption comprising a safe and effective amount of an anti-oxidant. Preferably, the daily dose is from about 0.00001 g to about 1 g, more preferably from about 0.0001 g to about 0.5 g and, even more preferably, from about 0.0005 g to about 0.1 g, of the anti-oxidant.

Anti-oxidants such as ester-C+, ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox^{R}, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pidolate, nordihydroguaiaretic acid, bioflavonoids, lysine, methionine, proline, superoxide dismutase, silymarin, mixed carotenoids (i.e. beta-carotene, lutein, lycopene), tea extracts, grape skin/seed extracts, melanin, and rosemary extracts may be used. Preferred anti-oxidants are selected from tocopherol acetate and other esters of tocopherol, more preferably tocopherol acetate.

IV. Vitamins

The kit of the present invention may also include a product suitable for oral consumption comprising a safe and effective amount of a vitamin or pro-vitamin (e.g., beta-carotene). Preferably, the daily dose is from about 0.00001 g to about 1 g, more preferably from about 0.0001 g to about 0.5 g and, even more preferably, from about 0.0005 g to about 0.1 g, of the vitamin.

Examples of vitamins that are useful herein include vitamin A, vitamin B, vitamin C, vitamin D, vitamin E, vitamin K; their derivatives; and mixtures thereof. Additionally, combinations of two or more vitamins may be used.

a) Vitamin B

Examples of Vitamin B compounds useful herein include vitamin B₁, vitamin B₂, vitamin B₃, vitamin B₅, vitamin B₆, vitamin B₁₂, vitamin B₁₅, their derivatives, and mixtures thereof.

For Example, a safe and effective amount of a vitamin B₃ compound may be used. Vitamin B₃ compounds are particularly useful for regulating skin condition as described in co-pending U.S. application Ser. No. 08/834,010, filed Apr. 11, 1997 (corresponding to international publication WO 97/39733 A1, published Oct. 30, 1997). When vitamin B₃ compounds are present in the kit of the instant invention, the daily dose is from about 0.00001 g to about 1 g, more preferably from about 0.0001 g to about 0.5 g and, even more preferably, from about 0.0005 g to about 0.1 g, of the vitamin B₃ compound.

As used herein, “vitamin B₃ compound” means a compound having the formula:

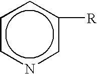

wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Examples of suitable vitamin B₃ compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

The vitamin compounds may be included as the substantially pure material, or as an extract obtained by suitable physical and/or chemical isolation from natural (e.g., plant) sources.

Also for example, a safe and effective amount of a vitamin B₆ compound may be used. The vitamin B₆ compounds useful herein include pyridoxine, esters of pyridoxine (e.g., pyridoxine tripalmitate), amines of pyridoxine (e.g., pyridoxamine), salts of pyridoxine (e.g., pyridoxine HCl) and derivatives thereof, including pyridoxamine, pyridoxal, pyridoxal phosphate, and pyridoxic acid. More preferably, the vitamin B₆ is selected from the group consisting of pyridoxine, esters of pyridoxine and salts of pyridoxine. Most preferably, the vitamin B₆ is pyridoxine HCl.

Vitamin B₆ can be synthetic or natural in origin and can be used as essentially as pure compounds or mixtures of compounds (e.g., extracts from natural sources or mixtures of synthetic materials). Vitamin B₆ is generally found in many foodstuffs, especially yeast, liver and cereals. As used herein, “vitamin B₆” includes isomers and tautomers of such and is commercially available from Sigma Chemical Co., St. Louis, Mo.

When vitamin B₆ compounds are present in the kit of the instant invention, the daily dose is from about 0.00001 g to about 1 g, more preferably from about 0.0001 g to about 0.5 g and, even more preferably, from about 0.0005 g to about 0.1 g, of the vitamin B₆ compound.

V. Micronutrient Metals

The kit of the present invention may also include a micronutrient metal. As used herein, “micronutrient metal” refers to metals that provide nutrients to a mammalian body that are necessary for proper total nutrition. Preferably, the daily dose is from about 0.00001 g to about 1 g, more preferably from about 0.0001 g to about 0.5 g and, even more preferably, from about 0.0005 g to about 0.1 g, of the micronutrient metal.

Examples of micronutrient metal that are useful herein include iron, zinc, selenium, copper, manganese; their derivatives; their salts; and mixtures thereof. Preferably, the micronutrient metal is copper, selenium, zinc and mixtures thereof.

VI. Pro-Biotics

The kit of the present invention may also include a pro-biotic. As used herein, “pro-biotic” refers to an organism that contributes to the health and balance of the intestinal tract. Preferably, the daily dose is from about 0.0001 g to about 1 g, more preferably from about 0.0001 g to about 0.5 g and, even more preferably, from about 0.0005 g to about 0.1 g, of the pro-biotic.

Examples of pro-biotics useful herein include Lactobacilli and Bifidobacteria. Additionally, combinations of two or more pro-biotics may be used.

VII. Pre-Biotics

The kit of the present invention may also include a pre-biotic. As used herein, “pre-biotic” refers to a substrate on which the bacteria (i.e., pro-biotic) feeds. Preferably, the daily dose is from about 0.00001 g to about 1 g, more preferably from about 0.0001 g to about 0.5 g and, even more preferably, from about 0.0005 g to about 0.01 g, of the pre-biotic.

Examples of pre-biotics useful herein include inulin and fructooligosaccharides. Additionally, combinations of two or more pre-biotics may be used.

VIII. Topical Skin and/or Hair Care Products

The kit of the present invention includes skin and/or hair care products that can be topically applied to mammalian keratinous tissue. Examples of such products that are useful herein include, but are not limited to, cosmetic creams, face and/or body moisturizers, facial and/or body cleansers, shampoos, conditioners, hair volumizers, hair sprays, hair shines, hair colorants, gels, pomades, lotions, and the like.

The topical compositions of the present invention may contain a variety of other ingredients that are conventionally used in given product types provided that they do not unacceptably alter the benefits of the invention.

The optional components, when incorporated into the composition, should be suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

1.) Desquamation Actives

A safe and effective amount of a desquamation active may be added to the compositions of the present invention, more preferably from about 0.01% to about 10%, even more preferably from about 0.5% to about 5%, also preferably from about 0.1% to about 2%, by weight of the composition. Desquamation actives enhance the skin appearance benefits of the present invention. For example, the desquamation actives tend to improve the texture of the skin (e.g., smoothness). One desquamation system that is suitable for use herein comprises sulfhydryl compounds and zwitterionic surfactants and is described in copending application Ser. No. 08/480,632, filed on Jun. 7, 1995 in the name of Donald L. Bissett, corresponding to PCT Application No. U.S. 95/08136, filed Jun. 29, 1995. Another desquamation system that is suitable for use herein comprises salicylic acid and zwitterionic surfactants and is described in copending patent application Ser. No. 08/554,944, filed on Nov. 13, 1995 as a continuation of Ser. No. 08/209,401, filed on Mar. 9, 1994 in the name of Bissett, corresponding to PCT Application No. 94/12745, filed Nov. 4, 1994, published May 18, 1995. Zwitterionic surfactants such as described in these applications are also useful as desquamatory agents herein, with cetyl betaine being particularly preferred.

2.) Anti-Acne Actives

The compositions of the present invention may comprise a safe and effective amount of one or more anti-acne actives. Examples of useful anti-acne actives include resorcinol, sulfur, salicylic acid, erythromycin, zinc, etc. Further examples of suitable anti-acne actives are described in further detail in U.S. Pat. No. 5,607,980, issued to McAtee et al, on Mar. 4, 1997.

3.) Anti-Wrinkle Actives/Anti-Atrophy Actives

The compositions of the present invention may further comprise a safe and effective amount of one or more anti-wrinkle actives or anti-atrophy actives. Exemplary anti-wrinkle/anti-atrophy actives suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids(e.g., salicylic acid, glycolic acid), keto acids (e.g., pyruvic acid), ascorbic acid (vitamin C), phytic acid, lipoic acid; lysophosphatidic acid, skin peel agents (e.g., phenol and the like), flavonoids (e.g., flavanones, chalcones, isoflavones, flavones, etc.), stilbenes, cinnamates, resveratrol, kinetin, zeatin, dimethylaminoethanol, peptides from natural sources (e.g., soy peptides), salts of sugar acids (e.g., Mn gluconate), terpene alcohols (e.g., farnesol), peptides and retinoids which enhance the keratinous tissue appearance benefits of the present invention, especially in regulating keratinous tissue condition, e.g., skin condition, and other vitamin B compounds (e.g., thiamine (vitamin B1), pantothenic acid (vitamin B5), carnitine (vitamin Bt), riboflavin (vitamin B2), cobalamine (vitamin B12), pangamic acid or diisopropylamine dichloroacetate (vitamin B15's), and their derivatives and salts (e.g., HCl salts or calcium salts)).

(a) Anti-Oxidants/Radical Scavengers

The compositions of the present invention may include a safe and effective amount of an anti-oxidant/radical scavenger. The anti-oxidant/radical scavenger is especially useful for providing protection against UV radiation that can cause increased scaling or texture changes in the stratum corneum and against other environmental agents, which can cause skin damage.

A safe and effective amount of an anti-oxidant/radical scavenger may be added to the compositions of the subject invention, preferably from about 0.1% to about 10%, more preferably from about 1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, hydroxy tyrosol, butylated hydroxy benzoic acids and their salts, 6-hydroxy-2,5,7,8-tetramethylchroman-2-carboxylic acid (commercially available under the tradename Trolox^{R}), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol sorbate. For example, the use of tocopherol sorbate in topical compositions and applicable to the present invention is described in U.S. Pat. No. 4,847,071, issued on Jul. 11, 1989 to Donald L. Bissett, Rodney D. Bush and Ranjit Chatterjee.

(b) Chelators

The compositions of the present invention may also comprise a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation that can contribute to excessive scaling or skin texture changes and against other environmental agents, which can cause skin damage.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995. Preferred chelators useful in compositions of the subject invention are furildioxime and derivatives.

(c) Flavonoids The compositions of the present invention may optionally comprise a flavonoid compound. Flavonoids are broadly disclosed in U.S. Pat. Nos. 5,686,082 and 5,686,367, both of which are herein incorporated by reference. Examples of flavonoids suitable for use in the present invention are flavanones selected from the group consisting of unsubstituted flavanones, mono-substituted flavanones, and mixtures thereof; chalcones selected from the group consisting of unsubstituted chalcones, mono-substituted chalcones, di-substituted chalcones, tri-substituted chalcones, and mixtures thereof; flavones selected from the group consisting of unsubstituted flavones, mono-substituted flavones, di-substituted flavones, and mixtures thereof; one or more isoflavones; coumarins selected from the group consisting of unsubstituted coumarins, mono-substituted coumarins, di-substituted coumarins, and mixtures thereof; chromones selected from the group consisting of unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents.

Preferred for use herein are flavones and isoflavones, in particular unsubstituted isoflavone, daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy), and mixtures thereof.

They can be synthetic materials or obtained as extracts from natural sources (e.g., plants). The naturally sourced material can also further be derivatized (e.g., an ester or ether derivative prepared following extraction from a natural source). Flavonoid compounds useful herein are commercially available from a number of sources, e.g., Indofine Chemical Company, Inc. (Somerville, N.J.), Steraloids, Inc. (Wilton, N.H.), and Aldrich Chemical Company, Inc. (Milwaukee, Wis.).

Mixtures of the above flavonoid compounds may also be used.

The herein described flavonoid compounds are preferably present in the instant invention at concentrations of from about 0.01% to about 20%, more preferably from about 0.1% to about 10%, and most preferably from about 0.5% to about 5%.

(d) Anti-Inflammatory Agents

A safe and effective amount of an anti-inflammatory agent may be added to the compositions of the present invention, preferably from about 0.01% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, including but not limited to, corticosteroids such as hydrocortisone..

A second class of anti-inflammatory agents, which is useful in the compositions, includes the nonsteroidal anti-inflammatory agents. The varieties of compounds encompassed by this group are well known to those skilled in the art. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974).

Specific non-steroidal anti-inflammatory agents useful in the composition invention include, but are not limited to, salicylates, flufenamic acid, etofenamate, aspirin, and mixtures thereof.

Finally, so-called “natural” anti-inflammatory agents are useful in methods of the present invention. Such agents may suitably be obtained as an extract by suitable physical and/or chemical isolation from natural sources (e.g., plants, fungi, by-products of microorganisms). For example, candelilla wax, alpha-bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Additional anti-inflammatory agents useful herein include allantoin and compounds of the Licorice (the plant genus/species Glycyrrhiza glabra) family, including glycyrrhetic acid, glycyrrhizic acid, and derivatives (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

The active component of these anti-inflammatory agents (e.g., biabolol, glycyrrhetinate esters) may also be obtained via extraction from natural sources or prepared synthetically.

(e) Anti-Cellulite Agents

The compositions of the present invention may also comprise a safe and effective amount of an anti-cellulite agent. Suitable agents may include, but are not limited to, xanthine compounds (e.g., caffeine, theophylline, theobromine, and aminophylline).

(f) Topical Anesthetics

The compositions of the present invention may also comprise a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

(g) Tanning Actives

The compositions of the present invention may comprise a tanning active. When present, it is preferable that the compositions comprise from about 0.1% to about 20%, more preferably from about 2% to about 7%, and most preferably from about 3% to about 6%, by weight of the composition, of dihydroxyacetone as an artificial tanning active.

Dihydroxyacetone, which is also known as DHA or 1,3-dihydroxy-2-propanone, is a white to off-white, crystalline powder. This material can be represented by the chemical formula C₃H₆O₃ and the following chemical structure:

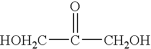

The compound can exist as a mixture of monomers and dimers, with the dimers predominating in the solid crystalline state. Upon heating or melting, the dimers break down to yield the monomers. This conversion of the dimeric form to the monomeric form also occurs in aqueous solution. Dihydroxyacetone is also known to be more stable at acidic pH values. See The Merck Index, Tenth Edition, entry 3167, p. 463 (1983), and “Dihydroxyacetone for Cosmetics”, E. Merck Technical Bulletin, 03-304 110, 319 897, 180 588.

(h) Skin Lightening Agents

The compositions of the present invention may comprise a skin lightening agent. When used, the compositions preferably comprise from about.0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, tranexamic acid, ascorbic acid and derivatives, e.g., magnesium ascorbyl phosphate or sodium ascorbyl phosphate or other salts of ascorbyl phosphate, ascorbyl glucoside, and the like. Other skin lightening materials suitable for use herein include undecylenoyl phenylalanine (Sepiwhite® from SEPPIC), aloesin, Actiwhite® (Cognis), Emblica® (??), and Azeloglicina (??). Skin lightening agents suitable for use herein also include those described in copending patent application Ser. No. 08/479,935, filed on Jun. 7, 1995 in the name of Hillebrand, corresponding to PCT Application No. U.S. 95/07432, filed Jun. 12, 1995; and copending patent application Ser. No. 08/390,152, filed on Feb. 24, 1995 in the names of Kalla L. Kvalnes, Mitchell A. DeLong, Barton J. Bradbury, Curtis B. Motley, and John D. Carter, corresponding to PCT Application No. U.S. 95/02809, filed Mar. 1, 1995, published Sep. 8, 1995.

(i) Antimicrobial and Antifungal Actives

The compositions of the present invention may comprise an antimicrobial or antifungal active. Such actives are capable of destroying microbes, preventing the development of microbes or preventing the pathogenic action of microbes. A safe and effective amount of an antimicrobial or antifungal active may be added to the present compositions, preferably, from about 0.001% to about 10%, more preferably from about 0.01% to about 5%, and most preferably from about 0.05% to about 2%.

Examples of antimicrobial and antifungal actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Preferred examples of actives useful herein include those selected from the group consisting of salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neocycin sulfate, and mixtures thereof.

(j) Sunscreen Actives

Exposure to ultraviolet light can result in excessive scaling and texture changes of the stratum corneum. Therefore, the compositions of the subject invention may optionally contain a sunscreen active. As used herein, “sunscreen active” includes both sunscreen agents and physical sunblocks. Suitable sunscreen actives may be organic or inorganic.

A wide variety of conventional sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one); 4-isopropyl-di-benzoylmethane; zinc oxide and titanium dioxide.

Of these, 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds, are preferred.

More preferred organic sunscreen actives useful in the compositions useful in the subject invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyidimethyl-p-aminobenzoic acid, octocrylene, zinc oxide, titanium dioxide, and mixtures thereof.

Especially preferred sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

A safe and effective amount of the sunscreen active is used, typically from about 1% to about 20%, more typically from about 2% to about 10% by weight of the composition. Exact amounts will vary depending upon the sunscreen chosen and the desired Sun Protection Factor (SPF).

(k) Conditioning Agents

The compositions of the present invention may comprise a conditioning agent selected from the group consisting of humectants, moisturizers, or skin conditioners. A variety of these materials can be employed and each can be present at a level of from about 0.01% to about 20%, more preferably from about 0.1% to about 10%, and most preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; and mixtures thereof. Also useful herein are the propoxylated glycerols described in U.S. Pat. No. 4,976,953, to Orr et al, issued Dec. 11, 1990.

Also useful are various C₁-C₃₀ monoesters and polyesters of sugars and related materials. These esters are derived from a sugar or polyol moiety and one or more carboxylic acid moieties. Such ester materials are further described in, U.S. Pat. No. 2,831,854, U.S. Pat. No. 4,005,196, to Jandacek, issued Jan. 25, 1977; U.S. Pat. No. 4,005,195, to Jandacek, issued Jan. 25, 1977, U.S. Pat. No. 5,306,516, to Letton et al, issued Apr. 26, 1994; U.S. Pat. No. 5,306,515, to Letton et al, issued Apr. 26, 1994; U.S. Pat. No. 5,305,514, to Letton et al, issued Apr. 26, 1994; U.S. Pat. No. 4,797,300, to Jandacek et al, issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985.

Preferably, the conditioning agent is selected from the group consisting of glycerol, urea, guanidine, sucrose polyester, and combinations thereof.

(l) Thickening Agent (Including Thickeners and Gelling Agents)

The compositions of the present invention can comprise one or more thickening agents, preferably from about 0.1 % to about 5%, more preferably from about 0.1 % to about 3%, and most preferably from about 0.25% to about 2%, by weight of the composition.

Nonlimiting classes of thickening agents include those selected from the group consisting of:

(i) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer thickeners useful herein are those selected from the group consisting of carbomers, acrylates/C₁₀-C₃₀ alkyl acrylate crosspolymers, and mixtures thereof.

(ii) Crosslinked Polyacrylate Polymers

The compositions of the present invention can optionally comprise crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. No.5,100,660, to Hawe et al, issued Mar. 31, 1992; U.S. Pat. No.4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Patent No.4,599,379 to Flesher et al issued Jul. 8, 1986; and EP 228,868, to Farrar et al, published Jul. 15, 1987.

(iii) Polyacrylamide Polymers

The compositions of the present invention can optionally comprise polyacrylamide polymers, especially nonionic polyacrylamide polymers including substituted branched or unbranched polymers. Most preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

(iv) Polysaccharides

A wide variety of polysaccharides are useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.).

Other useful polysaccharides include scleroglucans comprising a linear chain of (1-3) linked glucose units with a (1-6) linked glucose every three units, a commercially available example of which is Clearogel™ CS11 from Michel Mercier Products Inc. (Mountainside, N.J.).

(v) Gums

Other thickening and gelling agents useful herein include materials that are primarily derived from natural sources. Nonlimiting examples of these gelling agent gums include materials selected from the group consisting of acacia, agar, algin, alginic acid, ammonium alginate, amylopectin, calcium alginate, calcium carrageenan, camitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Preferred compositions of the present invention include a thickening agent selected from the group consisting of carboxylic acid polymers, crosslinked polyacrylate polymers, polyacrylamide polymers, and mixtures thereof, more preferably selected from the group consisting of carboxylic acid polymers, polyacrylamide polymers, and mixtures thereof.

Methods for Regulating Mammalian Keratinous Tissue

The personal care kit of the present invention is useful for regulating mammalian keratinous tissue condition. Such regulation includes prophylactic and therapeutic regulation. For example, such regulating methods are directed to preventing and/or retarding the appearance of spider vessels and/or red blotchiness on mammalian skin, preventing and/or retarding the appearance of puffy eyes and/or dark circles under the eye of a mammal, desquamating mammalian skin, preventing and/or retarding sallowness of mammalian skin, preventing and/or retarding sagging of mammalian skin, preventing and/or retarding eczema, softening and/or smoothing lips, hair and nails of a mammal, preventing and/or relieving itch of mammalian skin, preventing and/or treating UV damage of hair, preventing and/or treating hair loss and/or other hair growth abnormalities (i.e., thinning of hair, increased brittleness), preventing and/or treating dullness, and preventing and/or treating dry, itchy scalp.

The kit of the present invention may be presented to a user or potential user (hereinafter “users”) in association with information which informs such users that use of the kit will provide one or more benefits, including, but not limited to, preventing and/or retarding the appearance of spider vessels and/or red blotchiness on mammalian skin, preventing and/or retarding the appearance of puffy eyes and/or dark circles under the eye of a mammal, desquamating mammalian skin, preventing and/or retarding sallowness of mammalian skin, preventing and/or retarding sagging of mammalian skin, preventing and/or retarding eczema, softening and/or smoothing lips, hair and nails of a mammal, and preventing and/or relieving itch of mammalian skin, and the like. Such information may also include instructions for use to obtain such benefits, e.g., including the method steps described above. By “in association with information” it is meant that the information is either directly printed on the packaging of the kit itself (including direct printing on the container per se or indirectly via a label or the like affixed to the container), or presented in a different manner including, but not limited to, a brochure, print advertisement, electronic advertisement and/or other advertisement, so as to communicate the information to a consumer of the composition. Such information may accordingly comprise words, pictures, and the like.

According to the present invention, regulating keratinous tissue condition involves orally consuming a product suitable for oral consumption, e.g., dietary supplements, of the present invention in combination with topically applying to the keratinous tissue of a mammal in need of treatment a safe and effective amount of a composition suitable for topical use of the present invention. In the present invention, the dietary supplements should be consumed up to three times a day. The product suitable for topical application should be used at least two times a day.

In a preferred embodiment, the dietary supplement is in the form of capsules, tablets, powders, liquids, or nutritional foodstuffs. The regimen of consumption will vary according to the form of the dietary supplement. For example, if the dietary supplement is in the form of a capsule or tablet, preferably, it is taken orally with each meal daily. More preferably, the capsule or tablet is taken with a meal in the morning, in the afternoon, and the evening of each day. For example, the capsule or tablet may be taken daily with breakfast, lunch, and dinner. More preferably, there is oral consumption of the capsule or tablet form of dietary supplements of the present invention over an extended period during the subject's lifetime, preferably three times a day for a period of at least about a week, more preferably three times a day for a period of at least about one month, even more preferably three times a day for at least about three months, even more preferably three times a day for at least about six months, and more preferably still three times a day for at least about one year. While benefits are obtainable after various maximum periods of use (e.g., five, ten or twenty years), it is preferred that oral consumption of the capsule or tablet form of dietary supplements of the present invention continues throughout the subject's lifetime.

In combination with the oral consumption of the dietary supplements of the present invention, a product suitable for topical application is chronically applied to the skin and/or hair. By “chronic topical application” is meant continued topical application of the composition twice a day over an extended period during the subject's lifetime, preferably twice a day for a period of at least about a week, more preferably twice a day for a period of at least about one month, even more preferably twice a day for at least about three months, even more preferably twice a day for at least about six months, and still more preferably still twice a day for at least about one year. While benefits are obtainable after various maximum periods of use (e.g., five, ten or twenty years), it is preferred that chronic application continues throughout the subject's lifetime. Typically applications would be on the order of at least about twice per day over such extended periods, however application rates can vary from about once per week up to about three times per day or more.

A wide range of quantities of the topical product of the present invention can be employed to provide a keratinous tissue appearance and/or feel benefit. Quantities of the topical product that are typically applied per application will vary according to the user's level of regulation desired, e.g., in light of the level of skin and/or hair damage present or expected to occur.

For example, the topical product of the present invention useful for regulating skin condition is preferably practiced by applying a composition in the form of a skin lotion, cream, gel, foam, emulsion, spray, conditioner, tonic, cosmetic, lipstick, foundation, nail polish, after-shave, or the like which is intended to be left on the skin or other keratin structure for some aesthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, most preferably for at least several hours, e.g., up to about 12 hours. Any part of the external portion of the face, hair, and/or nails can be treated, e.g., face, lips, under-eye area, eyelids, scalp, neck, torso, arms, hands, legs, fingernails, toenails, scalp hair, eyelashes, eyebrows, etc.

Another approach to regulating skin condition via the topical product of the present invention is to provide the topical product in the form of a patch as part of the kit. Such an approach is particularly useful for problem skin areas needing more intensive treatment (e.g., facial crows feet area, under eye area, and the like). The patch can be occlusive, semi-occlusive or non-occlusive. The patch can also include additional actives such as chemical initiators for exothermic reactions such as those described in PCT application WO 9701313 to Burkett et al. The patch is preferably left on the skin for a period of at least about 5 minutes, more preferably at least about 15 minutes, more preferably still at least about 30 minutes, even more preferably at least about 1 hour, most preferably at night as a form of night therapy.

For example, the topical product of the kit of the present invention useful for regulating hair condition is preferably practiced by conventional ways to provide the conditioning and damage protection benefits of the present invention. Such method of use depends upon the type of composition employed but generally involves application of a safe and effective amount of the product to the hair and allowed to remain on the hair. By “effective amount” is meant an amount sufficient enough to provide a hair conditioning benefit. In general, from about 1 g to about 50 g is applied to the hair on the scalp. For example, the composition can be applied to wet or damp hair prior to drying of the hair. After such compositions are applied to the hair, the hair is dried and styled in accordance with the desires of the user and in the usual ways of the user. Alternately, the product can be applied to dry hair, and the hair is then combed or styled in accordance with the desires of the user.

### EXAMPLE

### Kit 1

Product A (Fatty Acid Oral Supplement)

One Capsule, 1000 mg total

Capsule A is formulated according to the following:

| | |
|---|---|
| Calories | 10 |
| Polyunsaturated Fat | 359 mg |

| | |
|---|---|
| EPA | 120 mg |
| DHA | 100 mg |
| DPA | 24 mg |
| Other | 115 mg |

| | |
|---|---|
| Monounsaturated Fat | 260 mg |
| Saturated Fat | 311 mg |
| Cholesterol | 2.8 mg |
| Sodium | 0 |
| Carbohydrate | 0 |
| Protein | 0 |
| Other | 57.2 mg |

Product B (Topical Product)

Topical Product B can be selected from the group consisting of cosmetic creams, face and/or body moisturizers, facial and/or body cleansers, shampoos, conditioners, hair volumizers, hair sprays, hair shines, hair colorants, gels, pomades, lotions, and the like.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care kit comprising separate containers packaged together in a unitary form comprising:
a) a product suitable for oral consumption comprising one or more of:
i) a safe and effective amount of one or more essential fatty acids;
ii) a safe and effective amount of a sugar amine or a pharmaceutically acceptable salt thereof; and
iii) a safe and effective amount of an anti-oxidant.
b) a product suitable for topical application to the skin and/or hair wherein a) and b) are individually contained then packaged together in a unitary form.

2. The kit of claim 1 wherein the essential fatty acid is selected from the group consisting of omega-3 fatty acids, omega-6 fatty acids, and mixtures thereof.

3. The kit of claim 2 wherein the omega-3 fatty acid is selected from the group consisting of alpha-linolenic acid, stearidonic acid, eicosapentanoic acid, docosahexanoic acid, and mixtures thereof.

4. The kit of claim 2 wherein the omega-6 fatty acid is selected from the group consisting of linoleic acid, gamma-linolenic acid, arachidonic acid, and mixtures thereof.

5. The kit of claim 1 wherein the essential fatty acid is selected from the group consisting of EPA, DHA, and mixtures thereof.

6. The kit of claim 1 wherein the sugar amine of subparagraph a) is selected from the group consisting of glucosamine, derivatives of glucosamine, N-acetyl glucosamine, galactosamine, derivatives of galactosamine, N-acetyl galactosamine, mannosamine, derivatives of mannosamine, N-acetyl mannosamine, and mixtures thereof.

7. The kit of claim 6 wherein the sugar amine is glucosamine.

8. The kit of claim 1 wherein the anti-oxidant of subparagraph a) is selected from the group consisting of grape seed extract, ester-C+, beta-carotene, lycopene, lutein, vitamin E, vitamin C; their derivatives; their salts; and mixtures thereof.

9. The kit of claim 1 wherein the anti-oxidant of subparagraph a) is vitamin E.

10. The kit of claim 1 further comprising a vitamin selected from the group consisting of vitamin A, vitamin B, vitamin C, vitamin D, vitamin K; their derivatives; and mixtures thereof.

11. The kit of claim 10 wherein the vitamin B is selected from the group consisting of vitamin B₁, vitamin B₂, vitamin B₃, vitamin B₅, vitamin B₆, vitamin B₁₂, vitamin B₁₅, their derivatives, and mixtures thereof.

12. The kit of claim 10 further comprising a micronutrient metal selected from the group consisting of copper, iron, zinc, selenium, manganese, and mixtures thereof.

13. The kit of claim 12 wherein the micronutrient metal is selected from the group consisting of copper, selenium, manganese, and mixtures thereof.

14. The kit of claim 1 wherein the product suitable for topical application is selected form the group consisting of cosmetic creams, face and/or body moisturizers, facial and/or body cleansers, shampoos, conditioners, hair volumizers, hair sprays, hair shines, hair colorants, gels, pomades, lotions and the like.

15. A method of improving the condition of skin and/or hair by utilizing the personal care kit of claim 1 wherein said method comprises the steps of:
i) orally administering up to three times a day to a mammal the product of subparagraph a); and
ii) topically applying up to two times a day to said mammal the product of subparagraph b).

16. The method of claim 15 wherein the oral administration of step i) is combined with a meal.

17. A personal care article of manufacture comprising:
A) unit packages in the form of capsules, tablets, powders, liquids, or nutritional foodstuffs comprising (a) from about 1 g to about 3 g of an essential fatty acid, (b) from about 1 g to about 3 g of a sugar amine; and (c) from about 0.0005 g to about 0.1 g of an anti-oxidant; and
B) a product suitable for topical application to the skin and/or hair
C) written and/or graphic instructions indicating that the product of A) should be taken together up to three times per day with the product of B) which should be used at least once a day in conjunction with the product of A), in accordance with a daily regimen for improving the condition of mammalian skin and/or hair.

18. A method of convincing a consumer to follow a treatment regimen, said treatment regimen comprising the personal care article of manufacture of claim 17.

19. A method of convincing a consumer to follow a treatment regimen, said treatment regimen comprising the use of a product suitable for topical application to the skin and/or hair with additional written and/or graphic instructions indicating that the topical product should be used at least once a day in conjunction with an oral nutritional supplement comprising one or more of:
i) a safe and effective amount of one or more essential fatty acids;
ii) a safe and effective amount of a sugar amine or a pharmaceutically acceptable salt thereof; and
iii) a safe and effective amount of an anti-oxidant.

20. A method of convincing a consumer to follow a treatment regimen, said treatment regimen comprising the use of a product suitable for oral consumption comprising one or more of:
i) a safe and effective amount of one or more essential fatty acids;
ii) a safe and effective amount of a sugar amine or a pharmaceutically acceptable salt thereof; and
iii) a safe and effective amount of an anti-oxidant
with additional written and/or graphic instructions indicating that the oral product should be taken together up to three times per day with a product suitable for topical application to the skin and/or hair.
